(19) United States
(12) Patent Application Publication
(10) Pub. No.: US 2018/0201999 A1
(43) Pub. Date: Jul. 19, 2018

(54) IDENTIFICATION OF FACTORS FOR THE TREATMENT OF MITOCHONDRIAL AND AGE-RELATED DISEASES

(71) Applicant: CYTEGEN CORPORATION, Shorewood, WI (US)

(72) Inventors: Ronald Blake HILL, Milwaukee, WI (US); N. George UGRAS, Menlo Park, CA (US); Tomas A. PROLLA, Madison, WI (US)

(21) Appl. No.: 15/744,919

(22) PCT Filed: Jul. 22, 2016
(86) PCT No.: PCT/US2016/043700

§ 371 Date: Jan. 15, 2018

Related U.S. Application Data
(60) Provisional application No. 62/196,262, filed on Jul. 23, 2015.

(51) Int. Cl. C12Q 1/6883; G01N 33/50; G06F 19/18

(52) U.S. Cl. CPC C12Q 1/6883; G01N 33/5079; C12Q 2600/156; G06F 19/18; G01N 33/5088

(57) ABSTRACT

The present disclosure provides a method of identifying factors for the treatment of mitochondrial and age-related diseases and disorders. Identification of the factors can comprise, e.g., biochemical, genomic, transcriptomic, proteomic and metabolomic analyses. The factors include proteins (e.g., cytokines) whose production or secretion into the blood is stimulated by exercise. The factors can be developed into therapeutics for the treatment of mitochondrial and age-related diseases and disorders.

### CROSS REFERENCE TO RELATED APPLICATIONS

This application claims priority to and the benefit of U.S. Provisional Patent Application No. 62/196,262 filed on Jul. 23, 2015, whose entire disclosure is incorporated herein by reference for all purposes.

### BACKGROUND OF THE DISCLOSURE

Mitochondria are essential sub-cellular particles involved in a variety of processes, including conversion of nutrients such as carbohydrate and fat into cellular energy in the form of adenosine triphosphate (ATP). Furthermore, mitochondria are involved in cell signaling, cell differentiation and cell death, as well as control of the cell cycle and cell growth. Mitochondrial dysfunction and decay increase with age, may potentially stem from, e.g., oxidative damage to components of mitochondria and mutations to mitochondrial DNA, and may ultimately lead to a variety of diseases.

### SUMMARY OF THE DISCLOSURE

The present disclosure describes a method of identifying factors for the treatment of mitochondrial and age-related diseases and disorders, comprising identifying factors in a biological fluid or tissue sample obtained from an animal that has been subjected to exercise. Identification of the factors can comprise biochemical analysis, genomic analysis, microarray data analysis, transcriptomic analysis, meta-analysis of genomic databases, proteomic analysis, metabolomic analysis and statistical analysis. Identification of the factors can further comprise testing the factors in cell-based assays to evaluate their ability to enhance mitochondrial fitness and function. The factors include proteins (e.g., cytokines) whose production or secretion into the blood is induced by exercise and which circulate throughout the body and act systemically to maintain or promote mitochondrial fitness and cell vitality, including maintenance of stem cell and progenitor cell populations. The factors can be developed into therapeutics for the treatment of mitochondrial and age-related diseases and disorders, including without limitation cardiovascular diseases (e.g., heart disease), metabolic diseases (e.g., diabetes), and neurodegenerative diseases (e.g., Alzheimer's disease and Parkinson's disease).

### BRIEF DESCRIPTION OF THE DRAWINGS

A better understanding of features and advantages of the present disclosure will be obtained by reference to the following detailed description, which sets forth illustrative embodiments of the disclosure, and the accompanying drawings.

FIG. 1 illustrates various ways of identifying factors which are enriched in a biological fluid or tissue sample obtained from an exercised PolG-D257A (POLG) mouse and which can be used to treat a mitochondrial or age-related disease.

### DETAILED DESCRIPTION OF THE DISCLOSURE

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein may be employed in practicing the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

### I. Definitions

As used in the specification and the appended claims, the indefinite articles “a” and “an” and the definite article “the” can include plural referents as well as singular referents unless specifically stated otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

In some embodiments, the term “upon exercise” means during exercise or within a short period of time after the termination of an exercise session or regimen. In other embodiments, the term “upon exercise” means after a certain amount of exercise or after a certain amount of tissue development as a result of exercise.

### II. Method of Identifying Factors for Treatment of Mitochondrial and Age-Related Diseases

The present disclosure provides a method of identifying factors in biological samples that can be developed into therapeutics for the treatment of mitochondrial and age-related diseases and disorders. The method utilizes, e.g., an animal model of aging, exercise as a stimulus of mitochondrial fitness or function, and analysis of biochemical assays, genomics, transcriptomics, proteomics and/or metabolomics to identify factors produced or secreted by the animal upon exercise. Exercise can stimulate the production or secretion of factors (e.g., proteins, such as cytokines) that improve mitochondrial fitness or function and promote the health or regeneration of cells, stem cells and/or progenitor cells. For example, exercise can increase the production of secreted proteins (e.g., cytokines) that stimulate mitochondrial biogenesis and selective autophagy of mitochondria (mitophagy, an important mitochondrial quality-control mechanism), which eliminates damaged mitochondria (e.g., those having damaged mtDNA), enriches healthy mitochondria, and supports stem cells and progenitor cells, leading to rejuvenation of the animal.

Some embodiments of the disclosure relate to a method of identifying factors for the treatment of mitochondrial or age-related diseases or disorders, comprising identifying factors in a biological fluid or tissue sample obtained from an animal that has been subjected to exercise. In certain embodiments, the method comprises:

subjecting an animal to exercise;

obtaining a biological fluid or tissue sample from the animal; and

identifying factors in the sample.

In some embodiments, the animal is a genetically engineered mouse having a mutation that increases the frequency of errors in mitochondrial DNA (mtDNA) replication and/or causes premature aging. In certain embodiments, the genetically engineered mouse is a PolG-D257A mouse (PolG^{D257A/D257A} mouse having a homozygous knock-in D257A mutation in the highly conserved exonuclease proofreading domain of the sole mammalian mtDNA polymerase, DNA polymerase γ [PolG]). The D257A mutation impairs the proofreading ability of PolG, thereby increasing mtDNA damage. PolG-D257A mice have an increased frequency of errors in mtDNA replication and exhibit a profound aging syndrome, including premature aging, cardiac dysfunction, osteoporosis, kyphosis, anemia, weight loss, decreased subcutaneous fat, alopecia, reduced fertility and shortened lifespan. Endurance exercise, however, reverses the aging syndrome in PolG-D257A mice (including restoration of normal lifespan), improves mitochondrial function (including restoration of the activity of the mitochondrial electron transport chain enzyme COX IV), and reduces pathologies in most or all tissues (including the brain). The exercised mice are indistinguishable from wild-type (wt) mice phenotypically and histologically (e.g., restoration of cardiomyocyte morphology). In some embodiments, PolG-D257A mice are used as an animal model of human aging. In further embodiments, PolG-D257A mice are used as an animal model of a human mitochondrial or age-related disease or disorder, such as, e.g., a cardiovascular disease, a metabolic disease (e.g., diabetes) and/or a neurodegenerative disease.

In some embodiments, the exercise comprises endurance exercise, rigorous exercise or resistance exercise. In certain embodiments, the exercise comprises endurance or rigorous exercise of a PolG-D257A mouse on a treadmill at a speed of about 10-20 m/min for about 30 minutes to about 1 hour at least once, twice or thrice per week over a period of about 1 month, 2 months, 3 months, 4 months, 5 months or 6 months. In some embodiments, the exercise comprises endurance or rigorous exercise of a PolG-D257A mouse on a treadmill at a speed of about 15 m/min for about 45 minutes three times per week over a period of about 4, 5 or 6 months. Control animals (e.g., a wild-type mouse on a calorie-restricted diet) can also be subjected to the same exercise regime.

Continued challenge that provides a certain level of stress may also have anti-aging effect. In some embodiments, in addition to or alternative to being or having been subjected to exercise, the animal is or has been exposed to repetitive or continual mild stress.

A biological fluid sample and/or a tissue sample can be obtained from the animal that has been subjected to exercise (and/or exposed to stress, such as repetitive or continual mild stress). In certain embodiments, the biological fluid sample comprises blood, plasma or serum. In other embodiments, the biological fluid sample comprises lymph. In yet other embodiments, the biological fluid sample comprises cerebrospinal fluid. In still other embodiments, the biological fluid sample comprises sweat. In further embodiments, the biological fluid sample comprises a tissue homogenate. In certain embodiments, the biological fluid sample is obtained from the animal immediately or shortly following or during exercise (and/or exposure to stress, such as repetitive or continual mild stress). The same kind of biological fluid sample can also be obtained from the animal shortly before exercise (and/or exposure to stress, such as repetitive or continual mild stress) as a control. More than one kind of biological fluid sample can be obtained from the animal immediately or shortly following or during exercise (and/or exposure to stress, such as repetitive or continual mild stress), and optionally shortly before exercise (and/or exposure to stress, such as repetitive or continual mild stress) as a control.

In further embodiments, the tissue sample comprises a tissue active during exercise (e.g., a muscle tissue). In some embodiments, the tissue sample comprises a tissue of the brain, heart, lung, kidney, liver, pancreas, small or large intestine, gonad, body fat, skin, hair or skeletal muscle (e.g., extensor digitorum longus, soleus, quadriceps femoris or tibialis anterior), any other tissue that secretes small molecules (e.g., metabolites or steroids) or large molecules (e.g., polypeptides or proteins), or a tissue homogenate, or any combination thereof. A tissue homogenate may be regarded as a biological fluid sample or a tissue sample. In certain embodiments, the tissue sample is obtained from the animal post-mortem immediately or shortly following exercise (and/or exposure to stress, such as repetitive or continual mild stress), or after an overnight fast. More than one type of tissue sample can be obtained from the animal immediately or shortly following exercise (and/or exposure to stress, such as repetitive or continual mild stress), or after an overnight fast.

Identification of factors in the biological fluid sample and/or the tissue sample can involve any of a variety of methodologies. In some embodiments, identification of factors comprises biochemical analysis, genomic analysis, transcriptomic analysis, proteomic analysis or metabolomic analysis, or any combination thereof. In certain embodiments, identification of factors comprises biochemical, genomic, transcriptomic, proteomic and metabolomic analyses. In further embodiments, identification of factors comprises analysis of DNA (e.g., genomic DNA), RNA (e.g., total RNA or mRNA), proteins, activity of enzymes, or small molecules (e.g., metabolites and steroids), or any combination thereof, including all of the preceding. As an example, in some embodiments total RNA is extracted from a biological fluid sample and/or a tissue sample obtained from the test animal and any control animals, the total RNA is converted to double-stranded (ds) cDNA, the ds cDNA is labeled with biotin, the biotin-labeled cDNA is fragmented, the cDNA fragments are hybridized to a gene (DNA) chip, the hybridized gene chip is washed and stained with streptavidin-phycoerythrin, and the stained gene chip is read by an instrument (e.g., a gene-array scanner). As another example, in some embodiments mRNA is extracted from a biological fluid sample and/or a tissue sample obtained from the test animal and any control animals, the mRNA is converted to ds cDNA and the ds cDNA is amplified by real-time quantitative polymerase chain reaction (PCR) using gene-specific probes, or the mRNA is reverse-transcribed to cDNA by a reverse transcriptase and the cDNA is amplified by PCR using gene-specific probes in reverse transcription-PCR, to measure mRNA levels in the samples.

To determine global gene expression differences as a result of exercise (and/or exposure to stress, such as repetitive or continual mild stress), gene expression can be measured by, e.g., DNA microarray methods and RNA-Seq (RNA Sequencing) methods. Gene expression studies (gene expression profiling is also called transcriptomics) can be conducted with DNA microarrays, which contain thousands of DNA sequences (probes) that potentially match complementary sequences in a sample and create a profile of most, or all, transcripts expressed. Microarrays target the identification of known common alleles (e.g., single nucleotide polymorphisms [SNPs]). Unlike DNA microarrays, RNA-Seq can be employed to detect and evaluate transcripts of rare allele variants. Furthermore, the transcriptome of a cell continually changes, and RNA-Seq (aka Whole Transcriptome Shotgun Sequencing [WTSS]) utilizes next-generation sequencing (NGS) to reveal a snapshot of RNA presence and quantity from a genome at a given moment in time. NGS facilitates sequencing of the RNA transcripts in a cell, providing the ability to study alternative gene-spliced transcripts, post-transcriptional modifications, gene fusion, mutations/SNPs and changes in gene expression. RNA-Seq can be done to study different populations of RNA, including total RNA, mRNA, and non-coding RNA (e.g., small RNA [such as miRNA], tRNA and rRNA). Statistical analysis of gene expression data using, e.g., a Bayesian probability can be done to determine whether the mean expression level of a gene is statistically different between exercised and sedentary animals.

The biochemical, genomic, transcriptomic, proteomic and/or metabolomic analyses can be performed of any suitable subjects, including the test subject and any controls. As an example, if the test animal that has been subjected to exercise (and/or exposed to stress, such as repetitive or continual mild stress) is a PolG-D257A mouse, analysis of biochemical, genomic, transcriptomic, proteomic and/or metabolomic profiles of a biological fluid sample and/or a tissue sample can be done of the test animal as well as of a sedentary and unstressed PolG-D257A mouse, a sedentary and unstressed PolG-D257A mouse on a calorie-restricted diet, an exercised wild-type mouse (e.g., a C57BL/6J mouse), an exercised wild-type mouse on a calorie-restricted diet (caloric intake reduced by, e.g., about 25%), a sedentary and unstressed wild-type mouse, or a sedentary and unstressed wild-type mouse on a calorie-restricted diet, or any combination thereof, as control(s).

In certain embodiments, analyses of DNA, RNA, proteins, enzyme activity and small molecules (e.g., metabolites and steroids) are conducted of blood specimens (e.g., blood, plasma and serum) and specimens of the brain, heart, lung, kidney, liver, pancreas, intestine (large and/or small), gonad, body fat and skeletal muscle (e.g., extensor digitorum longus, soleus, quadriceps femoris and tibialis anterior) obtained from the test animal (e.g., an exercised PolG-D257A mouse) and any control animals (e.g., a sedentary PolG-D257A mouse, an exercised wild-type mouse on a calorie-restricted diet, and a sedentary wild-type mouse on a calorie-restricted diet).

In further embodiments, identification of factors comprises analysis of transcriptomics. Transcriptomic analysis can be performed using, e.g., DNA microarray and RNA-Seq technologies to compare the expression levels of genes in the biological fluid sample and/or the tissue sample obtained from the test animal and any control animals. Thus, transcriptomic analysis enables determination of differences in the global gene expression between, e.g., exercised PolG-D257A mice and sedentary PolG-D257A mice. In yet further embodiments, identification of factors comprises meta-analysis of genomic databases. In certain embodiments, identification of factors comprises identifying from DNA sequences factors (e.g., proteins, such as cytokines) in a biological fluid (e.g., blood) and/or in a tissue active during exercise (e.g., a muscle tissue), or factors (e.g., proteins, such as cytokines) produced or secreted by a tissue active during exercise (e.g., a muscle tissue). For example, homology search and analysis of genomic databases can be performed to identify genes encoding potential secreted proteins (e.g., cytokines) based on their homology to genes encoding known secreted proteins.

In certain embodiments, identification of factors comprises administering (e.g., injecting) blood, plasma or serum from exercised PolG-D257A mice to sedentary and unstressed PolG-D257A mice to determine whether the blood, plasma or serum from exercised mice can reverse the aging syndrome in sedentary mice (e.g., rescue the aging phenotype of the latter). For example, serum from exercised mice can be injected (e.g., intraperitoneally) into sedentary mice three times per week for about 8 weeks to mimic endurance exercise comprised of running on a treadmill three times per week. In further embodiments, identification of factors comprises identifying proteins (e.g., cytokines) enriched in blood, plasma or serum from exercised PolG-D257A mice, not sedentary and unstressed PolG-D257A mice, using biochemical, genomic, transcriptomic, proteomic or metabolomic analysis, or any combination thereof. For instance, differences in the levels of proteins in the blood, plasma or serum from exercised mice and sedentary mice can be detected and quantified by biochemical, gene expression, proteomic and statistical (e.g., Bayesian) analyses. For proteomics, mass spectrometry (e.g., top-down mass spec) can be employed to determine differences in the expression of secreted proteins (e.g., cytokines) upon endurance exercise of PolG-D257A mice. In top-down mass spec, label-free and intact proteoforms, including potentially proteoforms having different post-translational modifications, are identified and relative quantification is possible for proteins having a molecular weight under about 30 kDa, compared to the typical method involving metabolic labeling, protease digestion, identification of peptide fragments by mass spec, and computational reconstruction of these fragments to identify proteins.

In some embodiments, identification of factors comprises evaluating a variety of criteria. One criterion can be abundance: relevant factors (e.g., cytokines) are anticipated to be present at substantially higher concentrations in exercised PolG-D257A mice than sedentary PolG-D257A mice in order to act systemically. Another criterion can be redundancy: proteins whose gene expression data is poorly correlated can be disregarded. Factors (e.g., cytokines) secreted upon exercise (and/or exposure to stress, such as repetitive or continual mild stress) may act systemically and on a variety of tissues. Such factors may act on a variety of tissues via a common receptor, which could be used to identify such factors. Yet another criterion can be biological precedent: a factor implicated in aging or mitochondrial or stem-cell biology can be given a higher priority, but unknown, relevant factors can be identified by, e.g., bioinformatic analysis.

In additional embodiments, identification of factors comprises assaying the factors (e.g., cytokines produced or secreted by a tissue active during exercise [e.g., a muscle tissue]) in a cell-based assay to evaluate their ability to improve mitochondrial fitness (e.g., biogenesis and mitophagy) or function (e.g., respiration and oxidative phosphorylation [ATP biosynthesis]). Such a cell-based assay is a non-limiting example of a “biochemical analysis” that can be conducted to identify relevant factors. The factors can be tested in an assay utilizing human cells (e.g., HeLa cells) and/or in an assay utilizing non-human cells (e.g., murine cells, such as those derived from PolG-D257A mice). The factors tested can be contained in a crude sample obtained from a test or control animal, can be isolated or purified from such a sample, or can be produced by, e.g., recombinant expression. As an illustrative example, samples (e.g., serum) obtained from exercised and sedentary PolG-D257A mice can be tested on mouse embryonic fibroblast (MEF) cells derived from PolG-D257A mice to assess the ability of components of the samples to improve mitochondrial fitness or function. Components of the samples can then be separated or purified by, e.g., extraction, fractionation and/or chromatography, and the separated or purified components can be re-tested in the cell-based assay to identify active components. Alternatively, components of samples (e.g., serum) obtained from exercised and sedentary PolG-D257A mice can first be separated or purified, and the separated or purified components can be tested in the cell-based assay to identify components that improve mitochondrial fitness or function. Analysis (e.g., genomic analysis) of tissues from exercised and sedentary PolG-D257A mice may identify cell types (whether MEF or other cell type) derived from PolG-D257A mice which are most suitable for such a cell-based assay.

The factors can also be tested in cell models and animal models of aging and various diseases. In certain embodiments, different pluralities (combinations) of factors are tested in cell-based assays and animal models. In some embodiments, identification of factors comprises administering (e.g., injecting) the factors to sedentary and unstressed PolG-D257A mice to assess their ability to retard, curtail or reverse the aging syndrome or aging-related effects (e.g., decline or functional deficits). In certain embodiments, different pluralities (combinations) of factors are administered to sedentary and unstressed PolG-D257A mice. The factors can be administered to the mice via any suitable mode (e.g., parenterally, such as intramuscularly, subcutaneously, intravenously or intraperitoneally) and in a suitable dose and frequency (e.g., at least once daily).

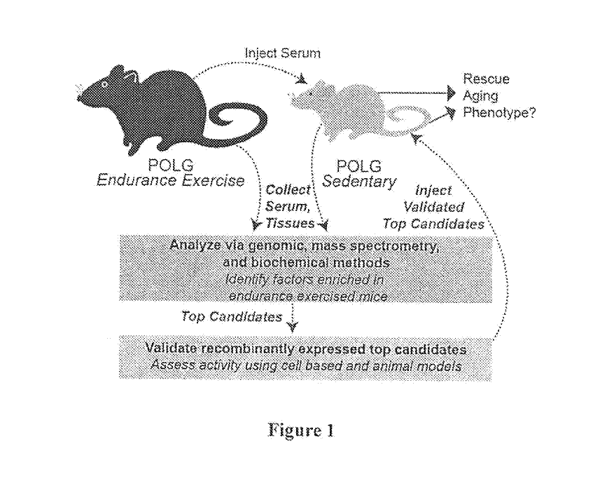

Various ways of identifying factors that are enriched in a biological fluid or tissue sample obtained from an exercised PolG-D257A mouse and that can be used to treat a mitochondrial or age-related disease or disorder have been described. Some of these ways are illustrated in FIG. 1, where “POLG” designates PolG-D257A.

Exercise (and/or exposure to stress, such as repetitive or continual mild stress) can improve mitochondrial health. Accordingly, in some embodiments one or more of the factors in the biological fluid sample and/or the tissue sample enhance mitochondrial fitness or function, enrich healthy mitochondria, or promote the elimination or replacement of damaged mitochondria, or any combination thereof.

Enhancement of mitochondrial fitness or function through, e.g., exercise can have beneficial effects on cells. Moreover, exercise can stimulate the production or secretion of factors (e.g., proteins, such as cytokines) that signal mitochondria to cause regeneration of healthy cells, stem cells and/or progenitor cells. Dysfunction of adult stem cells and progenitor cells can play an important role in aging. In some embodiments, one or more of the factors promote the health of cells, stem cells and/or progenitor cells, or the maintenance, rejuvenation or regeneration of cells, stem cells and/or progenitor cells.

Mitochondrial dysfunction or decay can result in damage to cells or tissues of, e.g., the brain, heart, kidney, liver or skeletal muscles, or the cardiovascular, endocrine, nervous or respiratory system. Such damage can cause diseases or disorders associated with aging. For example, mitochondrial dysfunction or decay can lead to neurodegenerative or neuromuscular diseases such as Alzheimer's disease or Parkinson's disease. Reactive byproducts of aerobic respiration in mitochondria, such as free radicals, potentially may over time cause damage (e.g., oxidative damage) to lipids, proteins, RNA and DNA in mitochondria and elsewhere in the cell, resulting in mitochondrial dysfunction and decay, apoptosis and age-related decline. Retardation of mitochondrial dysfunction or decay through, e.g., exercise may retard age-related decline. In certain embodiments, one or more of the factors whose production or secretion is induced by exercise (and/or exposure to stress, such as repetitive or continual mild stress) retard, curtail, reverse or prevent mitochondrial dysfunction, impairment, decay or disorders, and/or age-related decline, functional deficits or disorders.

The factors that can have beneficial effects on mitochondria and/or cells, and/or can have anti-aging effects, can be small molecules (e.g., metabolites or steroids) or large molecules (e.g., polypeptides or proteins). In some embodiments, the factors include proteins that have a molecular weight of no more than about 30, 25, 20, 15 or 10 kDa (e.g., no more than about 20 kDa). In certain embodiments, the protein factors include cytokines, including without limitation adipokines, chemokines, colony-stimulating factors, interferons, interleukins, monokines, myokines and lymphokines. Cytokines play an important role in intercellular communication and can act in an endocrine manner. In some embodiments, the cytokine factors include fractalkine [aka chemokine (C-X3-C motif) ligand 1 (CX3CL1)], growth differentiation factor 11 (GDF11), interleukin 10 (IL-10) and IL-15. In further embodiments, the factors include hormones, such as irisin and meteorin-like (Metml) protein. In yet further embodiments, the factors include growth factors. There may be some overlap in the terminology of cytokines, hormones and growth factors. For instance, growth differentiation factors (aka bone morphogenetic proteins) may be regarded as cytokines or growth factors.

The factors can be developed into therapeutics for the treatment of mitochondrion-associated diseases and disorders and aging-associated diseases and disorders. Mutations (including single nucleotide polymorphisms and deletions) in the sole mtDNA polymerase, DNA polymerase γ, cause a variety of diseases and disorders in humans, including without limitation metabolic diseases (e.g., diabetes), muscle diseases (e.g., mitochondrial myopathy), neuromuscular diseases (e.g., Charcot-Marie-Tooth disease [CMT], Parkinson's disease, ataxia neuropathy syndrome [ANS, including mitochondrial recessive ataxia syndrome {MIRAS} and sensory ataxia neuropathy dysarthria and ophthalmoplegia {SANDO}], and myoclonic epilepsy myopathy sensory ataxia [MEMSA]), neurodegenerative diseases (e.g., Alpers' disease [Alpers-Huttenlocher syndrome {AHS}] and Parkinson's disease), infantile myocerebrohepatopathy spectrum disorders, progressive external ophthalmoplegia (PEO) (including chronic PEO [cPEO], sporadic PEO [sPEO], autosomal dominant PEO [adPEO] and autosomal recessive PEO [arPEO]), tumors, cancers (e.g., testicular cancer), and male infertility. Factors (e.g., proteins, such as cytokines) that phenocopy exercise to overcome a defective mtDNA polymerase can be used to treat mitochondrial and age-related diseases and disorders.

In some embodiments, the mitochondrial and age-related diseases and disorders include diseases and disorders of the brain, eye, heart, liver, kidney, gonad, skeletal muscles, bones, joints, and cardiovascular, digestive, endocrine, respiratory, sensory (e.g., hearing) and central and peripheral nervous systems. In certain embodiments, the mitochondrial and age-related diseases and disorders include cardiovascular diseases (e.g., cardiac dysfunction, heart disease and atherosclerosis), hypertension, metabolic diseases (e.g., diabetes mellitus [e.g., type 2 diabetes] and Leigh's disease), diabetes and deafness, muscle diseases (e.g., mitochondrial myopathy), neuromuscular diseases (e.g., Charcot-Marie-Tooth disease [CMT], Parkinson's disease, ataxia neuropathy syndrome [including MIRAS and SANDO], and myoclonic epilepsy myopathy sensory ataxia [MEMSA]), neurodegenerative diseases (e.g., dementia [e.g., Alzheimer's disease], Alpers' disease, amyotrophic lateral sclerosis [ALS], Huntington's disease and Parkinson's disease), infantile myocerebrohepatopathy spectrum disorders, inflammatory diseases (e.g., arthritis, such as osteoarthritis [which can be caused by, e.g., diabetes]), osteoporosis (bone loss can be induced by, e.g., endocrine disorders, such as diabetes), kyphosis (hunchback), tumors, cancers (e.g., testicular cancer) (tumors and cancers can be caused by, e.g., age-related changes in the endocrine system), cataracts (lens proteins denature and degrade over time, which is accelerated by diseases such as diabetes and hypertension), Leber's hereditary optic neuropathy (LHON), Kearns-Sayre syndrome (KSS), progressive external ophthalmoplegia (PEO) (including cPEO, sPEO, adPEO and arPEO), hearing impairment and loss, anemia, weight loss, decreased subcutaneous fat, male infertility and alopecia (hair loss).

Additional embodiments of the disclosure relate to a method of developing therapeutics for the treatment of mitochondrial or age-related diseases or disorders, comprising:

identifying factors in a biological fluid or tissue sample obtained from an animal that has been subjected to exercise (and/or exposed to stress, such as repetitive or continual mild stress); and

developing the factors into therapeutics for the treatment of mitochondrial or age-related diseases or disorders.

In certain embodiments, the method of developing therapeutics for the treatment of mitochondrial or age-related diseases or disorders comprises:

subjecting an animal to exercise (and/or exposing the animal to stress, such as repetitive or continual mild stress);

obtaining a biological fluid or tissue sample from the animal;

identifying factors in the sample; and

developing the factors into therapeutics for the treatment of mitochondrial or age-related diseases or disorders.

### III. Representative Embodiments

The following embodiments of the disclosure are provided by way of example only:

1. A method of identifying factors for the treatment of mitochondrial or age-related diseases or disorders, comprising identifying factors in a biological fluid or tissue sample obtained from an animal that has been subjected to exercise.
2. The method of embodiment 1, wherein the animal is a genetically engineered mouse having a mutation that increases the frequency of errors in mitochondrial DNA (mtDNA) replication and/or causes premature aging.
3. The method of embodiment 2, wherein the genetically engineered mouse is a PolG-D257A mouse (PolG^{D257A/D257A} mouse having a homozygous knock-in D257A mutation in the exonuclease domain of DNA polymerase γ).
4. The method of any one of the preceding embodiments, wherein the exercise comprises endurance exercise, rigorous exercise or resistance exercise.
5. The method of any one of the preceding embodiments, wherein the biological fluid sample comprises blood, plasma, serum, lymph, cerebrospinal fluid, sweat or a tissue homogenate, or any combination thereof.
6. The method of any one of the preceding embodiments, wherein the tissue sample comprises a tissue active during exercise (e.g., a muscle tissue).
7. The method of any one of the preceding embodiments, wherein the tissue sample comprises a tissue of the brain, heart, lung, kidney, liver, pancreas, small or large intestine, gonad, body fat, skin, hair or skeletal muscle (e.g., extensor digitorum longus, soleus, quadriceps femoris or tibialis anterior), any other tissue that secretes small molecules (e.g., metabolites or steroids) or large molecules (e.g., polypeptides or proteins), or a tissue homogenate, or any combination thereof.
8. The method of any one of the preceding embodiments, wherein identifying factors in a biological fluid or tissue sample comprises biochemical analysis, genomic analysis, transcriptomic analysis, proteomic analysis or metabolomic analysis, or any combination thereof.
9. The method of any one of the preceding embodiments, wherein identifying factors in a biological fluid or tissue sample comprises analysis of DNA (e.g., genomic DNA), RNA (e.g., total RNA or mRNA), proteins, enzyme activity, or small molecules (e.g., metabolites and steroids), or any combination thereof.
10. The method of any one of the preceding embodiments, wherein identifying factors in a biological fluid or tissue sample comprises analysis of DNA microarray and/or RNA-Seq.
11. The method of any one of the preceding embodiments, wherein identifying factors in a biological fluid or tissue sample comprises meta-analysis of genomic databases.
12. The method of any one of the preceding embodiments, wherein identifying factors in a biological fluid or tissue sample comprises identifying from DNA sequences factors (e.g., proteins, such as cytokines) in a biological fluid (e.g., blood) or in a tissue active during exercise (e.g., a muscle tissue), or factors (e.g., proteins, such as cytokines) produced or secreted by a tissue active during exercise (e.g., a muscle tissue).
13. The method of any one of the preceding embodiments, wherein identifying factors in a biological fluid or tissue sample comprises assaying the factors in a cell-based assay to evaluate their ability to improve mitochondrial fitness (e.g., biogenesis and mitophagy) or function (e.g., respiration and oxidative phosphorylation [ATP biosynthesis]).
14. The method of any one of the preceding embodiments, wherein identifying factors in a biological fluid or tissue sample comprises administering the factors to sedentary PolG-D257A mice to assess their ability to retard, curtail or reverse aging syndrome or aging-related effects (e.g., decline or functional deficits).
15. The method of any one of the preceding embodiments, wherein one or more of the factors enhance mitochondrial fitness or function, enrich healthy mitochondria or promote the elimination or replacement of damaged mitochondria, or any combination thereof.
16. The method of any one of the preceding embodiments, wherein one or more of the factors promote the health of cells, stem cells or progenitor cells, or the maintenance, rejuvenation or regeneration of cells, stem cells or progenitor cells, or any combination thereof.
17. The method of any one of the preceding embodiments, wherein one or more of the factors retard, curtail, reverse or prevent mitochondrial dysfunction, impairment, decay or disorders, and/or age-related decline, functional deficits or disorders.
18. The method of any one of the preceding embodiments, wherein the factors include proteins.
19. The method of embodiment 18, wherein the protein factors include cytokines, hormones or growth factors, or any combination thereof.
20. The method of embodiment 19, wherein the protein factors include chemokine (C-X3-C motif) ligand 1 (CX3CL1), growth differentiation factor 11 (GDF11), interleukin 10 (IL-10), IL-15, irisin or meteorin-like (Metml) protein, or any combination thereof.
21. The method of any one of the preceding embodiments, wherein the mitochondrial or age-related diseases or disorders include diseases or disorders of the brain, eye, heart, liver, kidney, gonad, skeletal muscles, bones, joints, and cardiovascular, digestive, endocrine, respiratory, sensory (e.g., hearing) and central and peripheral nervous systems.
22. The method of any one of the preceding embodiments, wherein the mitochondrial or age-related diseases or disorders include cardiovascular diseases (e.g., cardiac dysfunction, heart disease and atherosclerosis), hypertension, metabolic diseases (e.g., diabetes mellitus [e.g., type 2 diabetes] and Leigh's disease), diabetes and deafness, muscle diseases (e.g., mitochondrial myopathy), neuromuscular diseases (e.g., Charcot-Marie-Tooth disease [CMT], Parkinson's disease, ataxia neuropathy syndrome [ANS, including mitochondrial recessive ataxia syndrome {MIRAS} and sensory ataxia neuropathy dysarthria and ophthalmoplegia {SANDO}], and myoclonic epilepsy myopathy sensory ataxia [MEMSA]), neurodegenerative diseases (e.g., dementia [e.g., Alzheimer's disease], Alpers' disease [Alpers-Huttenlocher syndrome {AHS}], amyotrophic lateral sclerosis [ALS], Huntington's disease and Parkinson's disease), infantile myocerebrohepatopathy spectrum disorders, inflammatory diseases (e.g., arthritis, such as osteoarthritis), osteoporosis, kyphosis (hunchback), tumors, cancers (e.g., testicular cancer), cataracts, Leber's hereditary optic neuropathy (LHON), Kearns-Sayre syndrome (KSS), progressive external ophthalmoplegia (PEO) (including chronic PEO [cPEO], sporadic PEO [sPEO], autosomal dominant PEO [adPEO] and autosomal recessive PEO [arPEO]), hearing impairment and loss, anemia, weight loss, decreased subcutaneous fat, male infertility and alopecia (hair loss).
23. The method of any one of the preceding embodiments, wherein the animal has further been exposed to repetitive or continual mild stress.
24. The method of any one of the preceding embodiments, further comprising developing the factors into therapeutics for the treatment of mitochondrial or age-related diseases or disorders.
25. A method of developing therapeutics for the treatment of mitochondrial or age-related diseases or disorders, comprising:

identifying factors in a biological fluid or tissue sample obtained from an animal that has been subjected to exercise; and

developing the factors into therapeutics for the treatment of mitochondrial or age-related diseases or disorders.

26. The method of embodiment 25, which comprises the method of any one of embodiments 1 to 24.

It is understood that, while particular embodiments have been illustrated and described, various modifications may be made thereto and are contemplated herein. It is also understood that the disclosure is not limited by the specific examples provided herein. The description and illustration of embodiments and examples of the disclosure herein are not intended to be construed in a limiting sense. It is further understood that all aspects of the disclosure are not limited to the specific depictions, configurations or relative proportions set forth herein, which may depend upon a variety of conditions and variables. Various modifications and variations in form and detail of the embodiments and examples of the disclosure will be apparent to a person skilled in the art. It is therefore contemplated that the disclosure also covers any and all such modifications, variations and equivalents.

What is claimed is:

1. A method of identifying factors for the treatment of mitochondrial or age-related diseases or disorders, comprising identifying factors in a biological fluid or tissue sample obtained from an animal that has been subjected to exercise.

2. The method of claim 1, wherein the animal is a genetically engineered mouse having a mutation that increases the frequency of errors in mitochondrial DNA (mtDNA) replication and/or causes premature aging.

3. The method of claim 2, wherein the genetically engineered mouse is a PolG-D257A mouse (PolG^{D257A/D257A} mouse having a homozygous knock-in D257A mutation in the exonuclease domain of DNA polymerase γ).

4. The method of any one of the preceding claims, wherein the exercise comprises endurance exercise, rigorous exercise or resistance exercise.

5. The method of any one of the preceding claims, wherein the biological fluid sample comprises blood, plasma, serum, lymph, cerebrospinal fluid, sweat or a tissue homogenate, or any combination thereof.

6. The method of any one of the preceding claims, wherein the tissue sample comprises a tissue active during exercise (e.g., a muscle tissue).

7. The method of any one of the preceding claims, wherein the tissue sample comprises a tissue of the brain, heart, lung, kidney, liver, pancreas, small or large intestine, gonad, body fat, skin, hair or skeletal muscle (e.g., extensor digitorum longus, soleus, quadriceps femoris or tibialis anterior), any other tissue that secretes small molecules (e.g., metabolites or steroids) or large molecules (e.g., polypeptides or proteins), or a tissue homogenate, or any combination thereof.

8. The method of any one of the preceding claims, wherein identifying factors in a biological fluid or tissue sample comprises biochemical analysis, genomic analysis, transcriptomic analysis, proteomic analysis or metabolomic analysis, or any combination thereof.

9. The method of any one of the preceding claims, wherein identifying factors in a biological fluid or tissue sample comprises analysis of DNA (e.g., genomic DNA), RNA (e.g., total RNA or mRNA), proteins, enzyme activity, or small molecules (e.g., metabolites and steroids), or any combination thereof.

10. The method of any one of the preceding claims, wherein identifying factors in a biological fluid or tissue sample comprises analysis of DNA microarray and/or RNA-Seq.

11. The method of any one of the preceding claims, wherein identifying factors in a biological fluid or tissue sample comprises meta-analysis of genomic databases.

12. The method of any one of the preceding claims, wherein identifying factors in a biological fluid or tissue sample comprises identifying from DNA sequences factors in a biological fluid or in a tissue active during exercise, or factors produced or secreted by a tissue active during exercise.

13. The method of any one of the preceding claims, wherein identifying factors in a biological fluid or tissue sample comprises assaying the factors in a cell-based assay to evaluate their ability to improve mitochondrial fitness (e.g., biogenesis and mitophagy) or function (e.g., respiration and oxidative phosphorylation [ATP biosynthesis]).

14. The method of any one of the preceding claims, wherein identifying factors in a biological fluid or tissue sample comprises administering the factors to sedentary PolG-D257A mice to assess their ability to retard, curtail or reverse aging syndrome or aging-related effects (e.g., decline or functional deficits).

15. The method of any one of the preceding claims, wherein one or more of the factors enhance mitochondrial fitness or function, enrich healthy mitochondria or promote the elimination or replacement of damaged mitochondria, or any combination thereof.

16. The method of any one of the preceding claims, wherein one or more of the factors promote the health of cells, stem cells or progenitor cells, or the maintenance, rejuvenation or regeneration of cells, stem cells or progenitor cells, or any combination thereof.

17. The method of any one of the preceding claims, wherein one or more of the factors retard, curtail, reverse or prevent mitochondrial dysfunction, impairment, decay or disorders, and/or age-related decline, functional deficits or disorders.

18. The method of any one of the preceding claims, wherein the factors include proteins.

19. The method of claim 18, wherein the protein factors include cytokines, hormones or growth factors, or any combination thereof.

20. The method of claim 19, wherein the protein factors include chemokine (C-X3-C motif) ligand 1 (CX3CL1), growth differentiation factor 11 (GDF11), interleukin 10 (IL-10), IL-15, irisin or meteorin-like (Metml) protein, or any combination thereof.

21. The method of any one of the preceding claims, wherein the mitochondrial or age-related diseases or disorders include diseases or disorders of the brain, eye, heart, liver, kidney, gonad, skeletal muscles, bones, joints, and cardiovascular, digestive, endocrine, respiratory, sensory (e.g., hearing) and central and peripheral nervous systems.

22. The method of any one of the preceding claims, wherein the mitochondrial or age-related diseases or disorders include cardiovascular diseases (e.g., cardiac dysfunction, heart disease and atherosclerosis), hypertension, metabolic diseases (e.g., diabetes mellitus [e.g., type 2 diabetes] and Leigh's disease), diabetes and deafness, muscle diseases (e.g., mitochondrial myopathy), neuromuscular diseases (e.g., Charcot-Marie-Tooth disease [CMT], Parkinson's disease, ataxia neuropathy syndrome [ANS, including mitochondrial recessive ataxia syndrome {MIRAS} and sensory ataxia neuropathy dysarthria and ophthalmoplegia {SANDO}], and myoclonic epilepsy myopathy sensory ataxia [MEMSA]), neurodegenerative diseases (e.g., dementia [e.g., Alzheimer's disease], Alpers' disease [Alpers-Huttenlocher syndrome {AHS}], amyotrophic lateral sclerosis [ALS], Huntington's disease and Parkinson's disease), infantile myocerebrohepatopathy spectrum disorders, inflammatory diseases (e.g., arthritis, such as osteoarthritis), osteoporosis, kyphosis (hunchback), tumors, cancers (e.g., testicular cancer), cataracts, Leber's hereditary optic neuropathy (LHON), Kearns-Sayre syndrome (KSS), progressive external ophthalmoplegia (PEO) (including chronic PEO [cPEO], sporadic PEO [sPEO], autosomal dominant PEO [adPEO] and autosomal recessive PEO [arPEO]), hearing impairment and loss, anemia, weight loss, decreased subcutaneous fat, male infertility and alopecia (hair loss).

23. The method of any one of the preceding claims, wherein the animal has further been exposed to repetitive or continual mild stress.

24. The method of any one of the preceding claims, further comprising developing the factors into therapeutics for the treatment of mitochondrial or age-related diseases or disorders.

25. A method of developing therapeutics for the treatment of mitochondrial or age-related diseases or disorders, comprising:
identifying factors in a biological fluid or tissue sample obtained from an animal that has been subjected to exercise; and
developing the factors into therapeutics for the treatment of mitochondrial or age-related diseases or disorders.

26. The method of claim 25, which comprises the method of any one of claims 1 to 24.
